(19) United States
(12) Patent Application Publication
(10) Pub. No.: US 2013/0324487 A1
(43) Pub. Date: Dec. 5, 2013

(54) Cyanidin-3-glucoside and Methods for Using the Same

(71) Applicant: Tim Patterson, Colorado Springs, CO (US)

(72) Inventor: Tim Patterson, Colorado Springs, CO (US)

(73) Assignee: MONSTEROPS LLC, Colorado Springs, CO (US)

(21) Appl. No.: 13/487,120

(22) Filed: Jun. 1, 2012

(51) Int. Cl. A61K 31/7048; A61P 3/04

(52) U.S. Cl. 514/27

(57) ABSTRACT

The present invention provides diet supplement compositions comprising cyanidin-3-glucoside (C3G) or C3G source or extracts and methods for using the same to aid in bodily fat loss.

### FIELD OF THE INVENTION

The present invention relates to diet supplement compositions comprising cyanidin-3-glucoside (C3G) or C3G source or extracts and methods for using the same to aid in bodily fat loss.

### BACKGROUND OF THE INVENTION

Obesity attributes to a wide range of health problems and has become an increasingly widespread, especially in the U.S. Obesity is a condition resulting from excessive body fat. One of the main contributing factors in obesity is overeating. The excess food (or energy therefrom) is then stored as fat. Obesity is believed to be one of the major contributing factors in hypertension, coronary heart disease, type 2 diabetes mellitus, stroke and even some forms of cancer.

Therefore, in order to reduce these risks, it is desirable to reduce stored body fat.

### SUMMARY OF THE INVENTION

Some aspects of the present invention provide diet supplement compositions comprising cyanidin-3-glucoside (C3G) or C3G source or extracts and methods for using the same, in particular in aiding bodily fat loss.

In some embodiments, the composition comprises C3G or its source or extract. When C3G source or its extract is used in the composition, the composition comprises C3G that is at about 20% purity or better. Still in other embodiments, the composition further comprises a microemulsification excipient. Within this embodiments, in some instances, the microemulsification excipient comprises lauroyl macrogol-32 glycerides. Such compositions are typically prepared by mixing these two components (and other optional components) by melting lauroyl macrogol-32 glycerides. Such mixtures can be prepared as capsules. In other embodiments, the purity of C3G ranges from about 35% to about 40%. Still in other embodiments, the C3G dosage is 125 mg per dosage unit (e.g., capsule), with about 600-650 mg of lauroyl macrogol-32 glycerides. Typically, oral administration of compositions of the invention ranges from about 2 to about 4 dosage units (e.g., capsules) at a time. In some instances, compositions of the invention are administered 3 to 4 times per day. Compositions of the invention can be used to enhanced body composition in, for example, adult humans and/or enhanced nutrient partitioning and/or enhanced athletic training or performance.

### DETAILED DESCRIPTION OF THE INVENTION

C3G of suitable purity can be obtained from ChromaDex. Suitable lauroyl macrogol-32 glycerides can be purchased as Gelucire 44/14 from Gattefosse Corp. Capsules suitable for oral nutraceuticals, dietary supplements, or pharmaceutical use are filled with C3G of 20% or greater purity, and more preferably 35% or greater purity, that has been heated and mixed with lauroyl macrogol-32 glycerides. A typical mixing and filling temperature ranges from about 44° C. to about 50° C.

It should be appreciated that other microemulsifying excipients similar to Gelucire 44/14 or lauroyl macrogol-32 glycerides can also be used. Accordingly, the scope of the invention is not limited to any particular microemulsifying excipients disclosed herein but includes any microemulsifying excipients that are known to one skilled in the art or are conventionally available as well as ones that are developed hereafter and are compatible with C3G. Typically, any microemulsifying excipients having a similar melting point range and HLB value can be used. In some instances, microemulsifying excipients with melting points in the range of from about 25° C. to about 47° C. and HLB value of 13-15 can be used. In other embodiments, excipient mixtures where at least 35% of the mixture comprises a microemulsifying agent with such HLB value, at least another 35% comprises a microemulsifying agent with an HLB value between 4 and 12, and the combined mixture having a melting point in the range of about 25° C. to about 47° C. can also be used.

In some embodiments, compositions of the invention is administered to the subject such that at least 200 mg per day and typically about 500 mg to about 2000 mg per day of C3G is provided to the subject. Such administration can be divided into 2 to 4 doses per day. Such amount is typically provided in capsules comprising about 100 to about 125 mg per dosage unit (e.g., capsule) of C3G. Compositions of the invention can be administered orally, with or without food, but typically prior to meals.

Compositions of the invention are useful in aiding in fat loss. In some embodiments, use of compositions of the invention aids in fat loss accompanied with concurrent substantial retention of lean muscle mass. Yet in other embodiments, use of compositions of the invention aids in fat loss accompanied with concurrent gain of lean muscle mass. Subjects who utilize compositions of the invention can be of normal health and/or normal body fat levels, or the individuals may have diabetes or metabolic syndrome, or may be obese or have unusually low body fat. Subjects desiring enhancement of athletic training or performance, or enhanced nutrient partitioning can also utilize compositions of the invention.

Administration of compositions of the invention can be ongoing or prolonged, or can be targeted, for example, with periods of one to four weeks of use followed by several weeks or months of disuse. Compositions and methods of the invention can be used to enhance body composition and/or enhance nutrient partitioning and/or enhance athletic training or performance either during the period of use, or during both the period of use and in following weeks where the formulation is not administered.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Testing on fifteen individuals has revealed that surprising degrees of fat loss with concurrent retention or gain of muscle mass were achieved with the formulation and method of administration taught, typically where the dosing comprises 3-4 of the above-described capsules taken 3-4 times per day, with total C3G dosing of 1,125-2,000 mg/day. These individuals included both athletes undergoing intensive training, and non-athletes in normal health with ordinary activity levels. The benefits were seen even in absence of metabolic disorder, diabetes, or obesity, even where body fat levels were already unusually low, and even where dietary intake was not restricted. In some cases, dietary intake was increased.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for aiding bodily fat loss in an individual, said method comprising the steps of administering to the individual a composition comprising cyanidin-3-glucoside (C3G) at a dose ranging from about 200 mg/day to about 2,000 mg/day.

2. The method of claim 1, wherein said composition is administered orally.

3. The method of claim 2, wherein said composition is a capsule.

4. The method of claim 3, wherein said method comprises administering from about 1 to about 4 capsules per dosing and more preferably 3-4 capsules per dosing.

5. The method of claim 4, wherein said method comprises administer the composition from about 1 to about 4 times per day and more preferably 3 or 4 times per day.

6. The method of claim 1, wherein said method comprises inducing body fat loss (fat loss) without reduced dietary intake, or with a smaller reduction of dietary intake than would otherwise be required in the absence of administering the composition.

7. The method of claim 1, wherein said method induces fat loss with concurrent retention or gain of lean body mass.

8. A diet supplement composition for aiding bodily fat loss comprising cyanidin-3-glucoside (C3G) and a microemulsification excipient.

9. The diet supplement composition of claim 8, wherein the weight of microemulsification excipient is at least twice the amount of C3G and more preferably at least 4 times.

10. The diet supplement composition of claim 8, wherein said microemulsification excipient comprises lauroyl macrogol-32 glycerides.

11. The diet supplement composition of claim 8, wherein said composition comprises from about 50 mg to about 250 mg of C3G per dosage unit and preferably about 125 mg per dosage unit.

12. A diet supplement composition comprising cyanidin-3-glucoside (C3G) source or extract and lauroyl macrogol-32 glyceride.

13. The diet supplement composition of claim 12, wherein said C3G source or extract is mixed with lauroyl macrogol-32 glyceride.

14. The diet supplement composition of claim 13, wherein said C3G source or extract and lauroyl macrogol-32 glyceride are melted at time of mixing.

15. The diet supplement composition of claim 12, wherein said composition is a capsule.

16. The diet supplement composition of claim 12, wherein the weight of lauroyl macrogol-32 glycerides present in said composition is at least twice the weight of C3G source or extract and more preferably at least 4 times.
